(19) United States
(12) Patent Application Publication
(10) Pub. No.: US 2020/0282034 A1
(43) Pub. Date: Sep. 10, 2020

(54) LIGHT-MEDIATED TREATMENTS OF METASTATIC CANCERS

(71) Applicant: The Board of Regents of the University of Oklahoma, Norman, OK (US)

(72) Inventors: Youngjae You, Edmond, OK (US); Sukyung Woo, Edmond, OK (US); Randle M. Gallucci, Edmond, OK (US)

(21) Appl. No.: 16/810,548

(22) Filed: Mar. 5, 2020

Related U.S. Application Data
(60) Provisional application No. 62/815,487, filed on Mar. 8, 2019.

(51) Int. Cl. A61K 39/00; A61K 39/39; A61K 41/00

(52) U.S. Cl. CPC A61K 39/0011; A61K 41/0057; A61K 41/0071; A61K 39/39

(57) ABSTRACT

A method of treating or mitigating metastatic cancer in a subject having a primary tumor, by providing a prodrug system, the prodrug system comprising (1) a prodrug comprising at least one functional moiety and at least one linker linked to the at least one functional moiety, wherein the at least one functional moiety is inactive when linked to the linker, and wherein the at least one linker is cleavable by singlet oxygen, and (2) a sensitizer which when exposed to an activator results in generation of singlet oxygen by the sensitizer, causing cleavage of the at least one linker thereby activating the at least one functional moiety, wherein the sensitizer optionally is linked to the functional moiety via the at least one linker, administering the prodrug system to the subject, and exposing the primary tumor to the activator causing activation of the functional moiety and death of at least a portion of the primary tumor. Also disclosed is a method of making a whole-cell cancer vaccine, and using the vaccine to treat or mitigate metastatic cancer in a subject.

### CROSS-REFERENCE TO RELATED APPLICATIONS

The present patent application incorporates by reference the entire provisional patent application identified by U.S. Ser. No. 62/815,487, filed on Mar. 8, 2019, and claims priority thereto under 35 U.S.C. 119(e).

### BACKGROUND

Breast cancer is the most frequently occurring cancer among all cancer types. Most patients (˜90%) are diagnosed before metastasis, but metastatic cancers often lead to death. Thus, it is critical to prevent recurrence and metastasis after the first treatment at the local and regional stages, as well as to effectively treat metastatic breast cancers. Chemotherapy is an essential treatment tool in breast cancers, as are hormonal agents and targeted therapy, which are not applicable for triple negative breast cancers (TNBCs). Such systemic therapeutics are vital treatment tools for metastatic breast cancers. However, systemic side effects accompany chemotherapy.

Fiber-optic guided visible and near infrared (IR) light “interstitial” therapy offers a minimally invasive therapy for local breast cancers. However, due to the thick human skin and relatively deep-seated tumors in breast cancers, externally illuminated light therapy has not been successfully adapted to breast cancer therapy. Thus, although approved for treatment of some cancer, photodynamic therapy (PDT) has not yet been approved by the FDA for breast cancer treatment. Recent advances in optical techniques (e.g., fiber-guided interstitial illumination) and imaging techniques (e.g., ultrasound imaging) have made PDT more applicable for local or recurrent chest wall breast cancers. It is well known that PDT stimulates the immune system to damage the illuminated cancers. More exciting is that the immune stimulation by PDT can also provide a systemic effect (a.k.a. the abscopal effect (AE)). However, up to now, the AE caused by PDT has not been very consistent or reproducible.

One of the major limitations of immune therapy has been its low response rate. For example, overall response rate to immune checkpoint inhibitor PD-1/PD-L1 monotherapy was 19% in TNBC patients with PD-L1 positive tumors. The combination of immune-modulating agents with traditional and new therapeutic regimens (such as chemotherapy, radiotherapy, and PDT) have shown improved response compared with monotherapy.

It is to the advancement of the applicability of AE to clinical applications, including its use as a co-treatment with chemotherapy, radiotherapy, and immune therapy, that the present disclosure is directed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic of (A) a multifunctional prodrug for combination therapy by PDT and local chemotherapy, and how (B) Bystander Effects can effectively kill surviving cancer cells after PDT damage by the released drugs (lifetime of singlet oxygen (SO) is very short (sub-microsecond scale), thus, direct cell damage by SO occurs to cells with prodrugs only during the irradiation).

FIG. 2 shows a schematic of how (A) limited diffusion of ¹O₂ can be overcome by extended diffusion of the released drug (D), and (B) Transient damage from ¹O₂ can be overcome by sustained damage from D ([D] in vivo concentration is expected to decrease mainly due to dilution by circulating blood). (C) shows an illustration of experimental setup for the horizontal illumination of a well (a), and live cell images of wells 72 h after illumination: no prodrug treatment (b), pseudo prodrug Pc-(NCL-CA4)₂ 25 nM (c), and prodrug Pc-(L-CA4)₂, 25 nM (d). Illumination was by 690 nm diode laser, 100 mW/cm² (at the incident point of the well), 30 min. (Pc=fluorescent PS phthalocyanine, CA4=drug combretastain A-4, NCL=noncleavable linker, L=SO-cleavable linker).

FIG. 3. (A) Structure of prodrug FA-PEG-Pc-L-PTX (2K-PTX), and (B) pharmokinetic data of 2K-PTX in plasma, tumor, and muscle after iv inj. at 1 μmole/kg, rat. (FA=folic acid, PEG=polyethylene glycol chain, PTX=paclitaxel).

FIG. 4A shows a schematic of a physiologically-based pharmacokinetic (PBPK) model for the prodrug 2K-PTX.

FIG. 4B shows representations of a mouse tumor model (i) and a rat tumor model (ii) used for preliminary data. mW/cm²=power density at target in the external hv, and mW/cm²=power per unit length of cylindrical diffuser in the interstitial hv, where hv=quantity of light energy.

FIG. 4C shows predicted pharmacokinetic (PK) profiles of released PTX in tumor (at 10% or 90% cleavage yield) and experimental PTX PK profile in the rat tumor model (FIG. 4B(ii)).

FIG. 5 shows (A) a Kaplan-Meier chart of results in the small (4-6 mm) tumor model: control*, three treated groups (illuminated at 0.5, 9, or 48 h-post iv inj. of FA-PEG-Pc-L-PTX at 1 mole/kg with 690 nm diode laser, 75 mW/cm\hat{ }2, 30 min, treated only once on day 0), (B) images from the big (16 mm) tumor model on rat (Ba) picture of illumination, (Bb) ultrasound image-guided insertion of the fiber to the tumor, and (Bc) tumor growth curves of each rat: control* and treated (illuminated at 9 h-post iv injection of the prodrug at 1 mole/kg with 690 nm diode laser, 75 mW/cm², 30 min, treated only once on day 0). * control: no prodrug & no illumination. **Groups receiving only prodrug or only illumination did not show significant antitumor effects.

FIG. 6A shows a schematic of experimental models for the abscopal effect (AE) in (i) treatment of metastatic (non-illuminated in righthand mouse) tumor (T2), (ii) tumor re-challenge, and (iii) metastasis to lung. Rejection of re-challenged cancer cells after the 1^{st} tumor was cured by 2K-PTX treatment (using model Aii):

FIG. 6B shows cure results using the Big MAT B III large tumor model (FIG. 4Bii), (i) tumor size curves (same data as FIG. 5Bc), (ii) re-challenged on 30 days after the treatment, (iii) re-challenged on 90 days, (iv) control tumor inoculated to naïve rat.

FIG. 6C shows results using the small tumor model (FIG. 4Bi), (i) tumor size curves (DLI=9 h), (ii) re-challenged on 41 days (2 mice), 45 (1 mouse), and 60 days (1 mouse).

FIG. 7A shows a Minimal PBPK model for the treatment tumor model in FIG. 6Ai.

FIG. 7B shows reasonably expected time profile of immune responses in the illuminated tumor (T1) following a immune stimulation at t=0 for the model of FIG. 7A.

FIG. 7C shows predicted tumor growth patterns for the model of FIG. 6Ai (i) with various power of AE and (ii) with only late effector and both early (a) and late (b) effectors following treatment (↑) on day 0.

FIG. 8A. Antitumor effects of treatment with prodrug (2K-PTX). (A) Tumor model and schedule for treatment with prodrug (2K-PTX).

FIG. 8B shows individual tumor response curves of treated and un-treated tumors following the schedule of FIG. 8A. The second tumor (T2) was smaller than the treated tumor (T1) on day 0, following the treatment (1 treatment on day 0, 4 rats/group.

FIG. 8C shows initial responses of the un-treated tumors of FIG. 8B.

FIG. 9A shows a pharmacodynamic profile of neutrophils in blood, following the treatment with the prodrug [rat, 16 mm MAT B III s.c. tumor; 2K-PTX, 1 mole/kg, DLI=9 h, 75 mW/cm, 30 min, interstitial illumination, 1 treatment on day 0; 3 (2 measurement/rat) rats/group] to treated and untreated tumors.

FIG. 9B shows a pharmacodynamic profile of eosinophils in blood, following the treatment with the prodrug [rat, 16 mm MAT B III s.c. tumor; 2K-PTX, 1 mole/kg, DLI=9 h, 75 mW/cm, 30 min, interstitial illumination, 1 treatment on day 0; 3 (2 measurement/rat) rats/group] to treated and untreated tumors.

FIG. 10A is an illustration of a two tumor model and treatment schedule (s.c., colon 26 cells on Balb/c mice) including treatment with anti-CTLA-4 (CTLA-4=cytotoxic T-lymphocyte-associated protein 4).

FIG. 10B shows an enhanced AE on the un-treated tumor (T2) by anti-CTLA-4 with DLI=9 h (T1 tumors were all cured). Numbers of mice per group with anti-CTLA-4 were 2.

FIG. 10C shows an enhanced AE on the treated tumor (T1) by anti-CTLA-4 with DLI=48 h. Number of mice per group with anti-CTLA-4 was 1.

### DETAILED DESCRIPTION

In at least certain embodiments, the present disclosure is directed to the use of a multifunctional prodrug platform, which is controllable by the use of an activator (e.g., illumination with near infrared (NIR) light), to not only provide drug delivery to the disease target, but also to utilize bystander effects to kill neighboring and metastatic cancer cells, while minimizing side effects. The prodrug platform, which includes but is not limited to the prodrug scaffolds shown in U.S. Pat. Nos. 9,393,306, and 9,839,690, overcomes many of the limitations of conventional photo sensitizers.

The present disclosure includes at least two types of therapeutic strategies for treating systemic cancers, such as breast cancer, including but not limited to treatment with (1) a light-activated prodrug used locally but having systemic anticancer effects, and (2) a whole cell vaccine produced by obtaining cells obtained from the patient's tumors and killing them with the disclosed prodrug (wherein the vaccine is then administered to the subject for treating or mitigating metastatic cancer in the subject). In clinical settings, the first treatment mode may be used for treatment of metastatic and recurrent cancers, e.g., cancers in the chest wall and other superficial areas where one tumor is readily reachable for illumination either by external beam or a thin interstitial optical fiber. The whole cell vaccine may be used to treat any metastatic and pre-metastatic conditions, but it particularly for adjuvant settings (i.e., as an additional treatment to lessen the risk that cancer will return), where tumor burden is small, to replace adjuvant chemotherapy, thus avoiding systemic toxicity.

Results, as discussed in further detail below, were obtained using two rodent tumor models to demonstrate the systemic anticancer effects. Animals with a local tumor were cured by the prodrug treatment. Then, the animals were re-challenged (injection at different site) by the same cancer cells at various dates after the initial treatment (e.g., up to −90 days). In work described below, these prodrugs have shown complete long-term cure (>90 days) in mice with small tumors (4-6 mm in length, external beam illumination) and in large tumors in rats (16 mm in length, fiber-guided interstitial illumination). All the animals blocked the formation of tumor mass. It was concluded that the prodrug treatment activated T-cells that were memorized in the immune system, which blocked the formation of tumor mass. In certain experiments, two tumors were made in one animal (mouse or rat) at distinct locations. When one tumor was treated by the prodrug PDT treatment, the other tumor, which was not treated by PDT, either shrunk or was completed eliminated. Based on the response seen of the second tumors, it was concluded that the prodrug treatment induced an additional mechanism of treatment, other than T-cell activation.

In addition to above systemic anticancer effect from the prodrug monotherapy, we observed that its combination with immunotherapeutics (e.g., checkpoint inhibitors and others) synergistically improve the systemic anticancer effect. When mice with two tumors were first treated with our prodrug and then treated with anti-CTLA-4, the antitumor effect on the un-treated tumor improved. The systemic antitumor effect was shown not only on un-treated tumor but also on sub-optimally treated tumor. When the first tumor was treated with the prodrug at a low level, the first tumor was not cured. But in combination with anti-CTLA-4, the tumor was further damaged, and eventually eliminated.

Before further describing various embodiments of the compounds, compositions and methods of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure. Thus, while the compounds, compositions, and methods of the present disclosure have been described in terms of particular embodiments, it will be apparent to those of skill in the art that variations may be applied to the compounds, compositions and/or methods and in the steps or in the sequence of steps of the methods described herein without departing from the spirit, and scope of the inventive concepts described herein.

All patents, published patent applications, and non-patent publications mentioned in the specification or referenced in any portion of this application, including U.S. Pat. Nos. 9,393,306 and and 9,839,690, and provisional application U.S. Ser. No. 62/815,487, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately”, where used herein when referring to a measurable value such as an amount, a temporal duration, and the like, is meant to encompass, for example, variations of ±20%, or ±15%, or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 75% of the time, or at least 80% of the time, or at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

As used herein any reference to “we” as a pronoun herein refers generally to laboratory personnel or other contributors who assisted in the laboratory procedures and data collection and is not intended to represent an inventorship role by said laboratory personnel or other contributors in any subject matter disclosed herein.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds of the present disclosure may be combined with one or more pharmaceutically-acceptable excipients, including carriers, vehicles, diluents, and adjuvents which may improve solubility, deliverability, dispersion, stability, and/or conformational integrity of the compounds or conjugates thereof.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention. Non-limiting examples of modes of administration include oral, topical, retrobulbar, subconjunctival, transdermal, parenteral, subcutaneous, intranasal, intramuscular, intraperitoneal, intravitreal, and intravenous routes, including both local and systemic applications. In addition, the compositions of the present disclosure may be designed to provide delayed, controlled, extended, and/or sustained release using formulation techniques which are well known in the art.

The terms “therapeutic composition” and “pharmaceutical composition” refer to composition comprising a compound of the present disclosure (also referred to herein as an active agent) that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” or “mitigate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” by ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

Where used herein, the term “eliciting an immune response” or “inducing an immune response” means initiating, triggering, causing, enhancing, improving or augmenting any response of the immune system, for example, of either a humoral or cell-mediated nature. The initiation or enhancement of an immune response can be assessed using assays known to those skilled in the art including, but not limited to, antibody assays (for example ELISA assays), antigen specific cytotoxicity assays and the production of cytokines (for example ELISPOT assays).

The terms “cancer” and “cancerous” refer to or describe the physiological condition in mammals that is typically characterized by unregulated cell growth. Examples of cancers and tumors include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer. Metastatic cancer or metastatic cancer cells refers to cancerous tumors or cancerous cells which have spread away from the original or primary site of the cancer tumor to another site of the body, for example a cancer which has spread from a breast to a bone.

The terms “administration” and “administering”, as used herein will be understood to include all routes of administration known in the art, including but not limited to, oral, topical, transdermal, parenteral, subcutaneous, intranasal, mucosal, intramuscular, intraperitoneal, intravitreal and intravenous routes, including both local and systemic applications. In addition, the compositions of the present disclosure (and/or the methods of administration of same) may be designed to provide delayed, controlled or sustained release using formulation techniques which are well known in the art.

In certain non-limiting embodiments, the dosage of the prodrug or whole cell vaccine composition administered to a subject could be in a range of 1 μg per kg of subject body mass to 1000 mg/kg, or in a range of 5 μg per kg to 500 mg/kg, or in a range of 10 μg per kg to 300 mg/kg, or in a range of 25 μg per kg to 250 mg/kg, or in a range of 50 μg per kg to 250 mg/kg, or in a range of 75 μg per kg to 250 mg/kg, or in a range of 100 μg per kg to 250 mg/kg, or in a range of 200 μg per kg to 250 mg/kg, or in a range of 300 μg per kg to 250 mg/kg, or in a range of 400 μg per kg to 250 mg/kg, or in a range of 500 μg per kg to 250 mg/kg, or in a range of 600 μg per kg to 250 mg/kg, or in a range of 700 μg per kg to 250 mg/kg, or in a range of 800 μg per kg to 250 mg/kg, or in a range of 900 μg per kg to 250 mg/kg, or in a range of 1 mg per kg to 200 mg/kg, or in a range of 1 mg per kg to 150 mg/kg, or in a range of 2 mg per kg to 100 mg/kg, or in a range of 5 mg per kg to 100 mg/kg, or in a range of 10 mg compound per kg to 100 mg/kg, or in a range of 25 mg per kg to 75 mg/kg.

Vaccine preparations containing immunogenic compositions of the present disclosure may be used by administering said preparation via systemic or mucosal route or other suitable route. These administrations may include injection via the intramuscular, intraperitoneal, intradermal or subcutaneous routes; or via mucosal administration to the oral/alimentary, respiratory, genitourinary tracts. Although the vaccine may be administered as a single dose, components thereof may also be co-administered together at the same time or at different times. In addition to a single route of administration, two different routes of administration may be used. For example, (IM) or intradermally (ID) and intranasally (IN) or intradermally (ID).

In certain non-limiting embodiments, the content of the vaccine will typically be in the range 1-1000 μg, 5-500 μg, or in the range 10-100 μg. Following an initial vaccination, subjects may receive one or several booster immunizations adequately spaced. Vaccine preparation is generally described in Vaccine Design (“The subunit and adjuvant approach” (eds Powell M. F. & Newman M. J.) (1995) Plenum Press New York). A non-limiting description of encapsulation of the immunogenic composition and/or vaccine formulation within liposomes is described by Fullerton, U.S. Pat. No. 4,235,877.

In one aspect, the present disclosure includes a vaccine kit, comprising a vial containing an immunogenic composition of the disclosure, optionally in lyophilized form, and further comprising a vial containing an adjuvant. It is envisioned that in this aspect, the adjuvant will be used to reconstitute the lyophilized immunogenic composition.

In certain embodiments, generation of a protective immune response by the vaccine can be measured by the development of antibodies. The amounts of the vaccine preparation described herein that can form a protective immune response may be, in certain non-limiting embodiments, in a unit dosage form of about 0.001 μg to 100 mg per kg of body weight, 0.01 μg to 1 mg/kg of body weight, or about 0.1 μg to about 10 μg/kg body weight, for example, at an interval of about 1 to 6 weeks intervals between immunizations.

Functional moieties of the prodrugs of the present disclosure include, but are not limited to the following compounds: DNA cross-linking agents including DNA alkylating agents such as Altretamine and Busulfan; Nitrogen mustards such as Bendamustine, Chlorambucil, Cyclophosphamide, Ifosfamide, Mechlorethamine, Melphalan, and Thiotepa; Nitrosoureas such as Carmustine, Lomustine, and Streptozocin; Organoplatinum complexes such as Carboplatin, Cisplatin, Oxaliplatin, Picoplatin, and Satraplatin; Procarbazine and triazenes such as Dacarbazine, Procarbazine, and Temozolomide; Antimetabolites such as Antifolates, Methotrexate, Pemetrexed, Pralatrexate; DNA methyltransferase inhibitors such as Azacitidine, Decitabine, Nelarabine; DNA polymerase inhibitors such as Cladribine, Clofarabine, Cytarabine, Fludarabine, and Gemcitabine; Miscellaneous antimetabolites such as Hydroxyurea and Pentostatin; Pyrimidine antagonists and Capecitabine, Floxuridine, and Fluorouracil; Purine antagonists such as Mercaptopurine and Thioguanine; Histone deacetylase inhibitors such as Romidepsin and Vorinostat; Immunomodulators such as Lenalidomide and Thalidomide; antibiotics such as Bleomycin, Dactinomycin and Mitomycin; anticancer agents such as Arsenic trioxide and Bortezomib; Mitosis inhibitors such as Cabazitaxel, Docetaxel, Estramustine, Ixabepilone, Paclitaxel, Vinblastine, Vincristine, and Vinorelbine; Topoisomerase poisons such as Daunorubicin, Doxorubicin, Epirubicin, Etoposide, Idarubicin, Irinotecan, Mitoxantrone, Teniposide, Topotecan, and Valrubicin; Tyrosine kinase and related inhibitors such as Dasatinib, Erlotinib, Everolimus, Gefitinib, Imatinib, Lapatinib, Nilotinib, Sorafenib, Sunitinib, and Temsirolimus.

In certain embodiments, the functional moiety may be a biologically active moiety, such as but not limited to, small molecules, peptide, proteins, nucleotides (RNA, DNA, etc.) and the like, including therapeutic moieties. Therapeutic moieties may be any molecule capable of exhibiting a desired therapeutic effect. Non-limiting examples of inactive therapeutic moieties that may be utilized in accordance with the presently disclosure include pro-drugs and nano-drug-carriers (such as but not limited to, deactivated drugs encapsulated in liposomes, polymers, nanospheres, nanocapsules, micelles, dendrimeric structures, solid lipid nanoparticles, other nanostructures, micro-particles and other micro-structures, gel and solid formulations, fullerenes (such as but not limited to, buckyballs), combinations and derivatives thereof, and the like). Particular non-limiting examples described herein include Estron, Combretastatin A4, SN-38, and paclitaxel. In other embodiments, the functional moiety may be a detectable moiety. The detectable moiety may be any molecule capable of detection by a detection method. Non-limiting examples of detectable moieties include reporter molecules, imaging agents, and the like. Particular non-limiting examples of detectable moieties include fluorophores, MRI contrasting agents, enzymes, radioisotopes, sensitizing agents (used for ultrasound, photoacoustic imaging, radiography, and the like), and combinations and derivatives thereof.

The targeting/delivery moiety may be any molecule known in the art or otherwise contemplated herein that is capable of associating with a desired location/site, whereby the composition is targeted to the site, and the individual components thereof (including the activatable functional moiety) is thus delivered to the desired location/site. Non-limiting examples include antibodies, ligands, tumor markers, aptamers, PEG, albumin, tumor specific peptides, AFFIBODY® molecules (Affibody AB, Solna, Sweden), vitamins (such as but not limited to, folic acid), carbohydrates, hormones, low density lipoproteins (LDL), and the like, as well as combinations and derivatives thereof. The targeting/delivery moiety may bind to a receptor or other molecule exposed on the surface of a cell; optionally, the targeting/delivery moiety may pass through the cell membrane and bind to intracellular components. The present disclosure is further directed to methods of producing the compositions described herein above.

In non-limiting embodiments, the activator comprises irradiation with light in a range of from about 380 nm to about 1200 nm. Particular examples of ranges of light wavelength that may be utilized include a lower range limit of about 380 nm, about 400 nm, about 425 nm, about 450 nm, about 475 nm, about 500 nm, about 525 nm, about 550 nm, about 575 nm, about 600 nm, about 650 nm, about 675 nm, about 700 nm, about 725 nm, about 750 nm, about 775 nm, about 800 nm, about 825 nm, about 850 nm, and the like, and an upper range limit of about 1200 nm, about 1175 nm, about 1150 nm, about 1125 nm, about 1100 nm, about 1025 nm, about 1000 nm, about 975 nm, about 950 nm, about 925 nm, about 900 nm, about 875 nm, about 850 nm, about 825 nm, about 800 nm, about 775 nm, about 750 nm, and the like. In certain embodiments, short and mid-visible light, long visible light, and/or near IR light irradiation may be used as the activator.

In certain embodiments, the sensitizer (such as but not limited to, a photosensitizer) is positioned in close proximity to the linker, for example by being conjugated to the linker, or simply being in close physical, but unlinked, proximity to the linker. Exposure of the sensitizer to an activator results in generation of singlet oxygen by the sensitizer. Generation of singlet oxygen in close proximity to the linker results in cleavage of the linker tethered to the functional moiety, thus activating the functional moiety (when a targeting/delivery moiety is present in the composition, the activation of the functional moiety occurs at the desired delivery site). Selection of the sensitizer may be based upon the activator utilized. For example, when visible/near IR radiation is utilized as the activator, a photosensitizer is used; when luminescence is utilized as the activator, a combination of chemicals or a chemical and enzyme combination may be used; when light wavelengths longer than 800 nm are used, two photon absorbing photosensitizers (or two photon absorbers with photosensitizers) may be used. Non-limiting examples of photosensitizers that may be utilized include porphyrin, phthalocyanines, BODIPY (or aza-BODIPY)-type photosensitizers, chlorins, bacteriochlorins, any non-porphyrin-based photosensitizers, and combinations and derivatives thereof. In addition, non-limiting examples of other sensitizers include single-walled carbon nanotubes, conjugates of light harvesting materials (either via one or two photon absorption) and photosensitizer, nanoscintillator alone or with sensitizers, sonar sensitizers (used with ultrasound), radiosensitizers (used with ionization waves such as x-rays and γ-rays), MRI contrasting agents (used with long magnetic waves), and the like, as well as combinations and derivatives thereof.

The linker utilized herein may be any molecule capable of (1) being tethered to the inactive functional moiety, (2) being cleavable by singlet oxygen, (3) allowing for the direct or indirect activation of the functional moiety upon cleavage thereof, and (4) when a targeting/delivery moiety is present, linking the inactive functional moiety to the targeting/delivery moiety. Non-limiting examples of linkers that fall within the scope of the present disclosure include aminoacrylate, aminoacyltholate, aminoacrylamide, beta-aminoketone, and combinations and derivatives thereof, for example as shown in U.S. Pat. Nos. 9,393,306, and 9,839,690, which are expressly incorporated herein by reference.

Examples are provided herein. However, the present disclosure is to be understood to not be limited in its application to the specific experimentation, results, and laboratory procedures. Rather, the Examples are simply provided as one of various embodiments and are meant to be exemplary, not exhaustive. However, it is to be understood that the information contained therein is provided for the purpose of description, and the present disclosure is not limited to such exemplary information contained therein.

### EXPERIMENTAL

A Light-Activatable Prodrug Platform Offering Prodrug Design with Multi Functionality, Such as Dual Therapeutic Modality (PDT and Site-Specific Chemotherapy), Optical Imaging, and Targeting

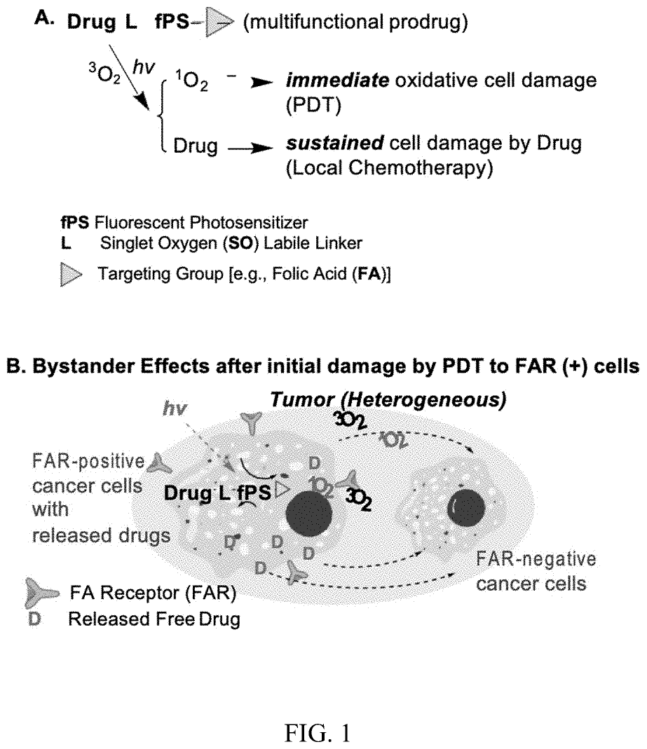

A goal of light-activated therapy, particularly for solid tumors, is a system that can be activated by long visible and near IR wavelengths (e.g., 600-800 nm) for deep tissue penetration. However, due to the low energy of such light, chemical linkers that can be directly cleaved by such light have not been available. The present drug platform utilizes a new linker (L) that can be cleaved by singlet oxygen (SO) generated by the illumination of the low energy light to photosensitizers. Conjugated together by the SO-cleavable linker L is a drug and a fluorescent photosensitizer (fPS) forming the prodrug (Drug-L-fPS), e.g., see FIG. 1A). Targeting functionality can be added to the core or through formulation techniques, either conjugating a targeting moiety, e.g., folic acid, or by formulating nanosized micelles. Two prodrug formulations comprising paclitaxel (PTX) and combretastatin A4 (CA4) using silicon phthalocyanine (Pc) as the photosensitizer fPS were formulated and utilized below for experimental purposes.

Site-Specifically Released Anticancer Drugs Kill Surviving Cells from the Initial PDT Damage

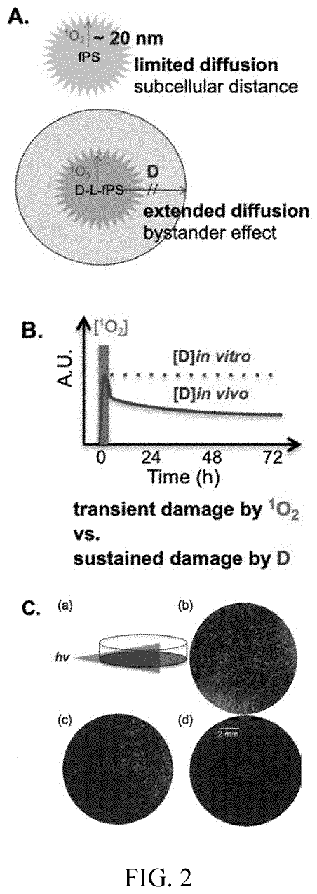

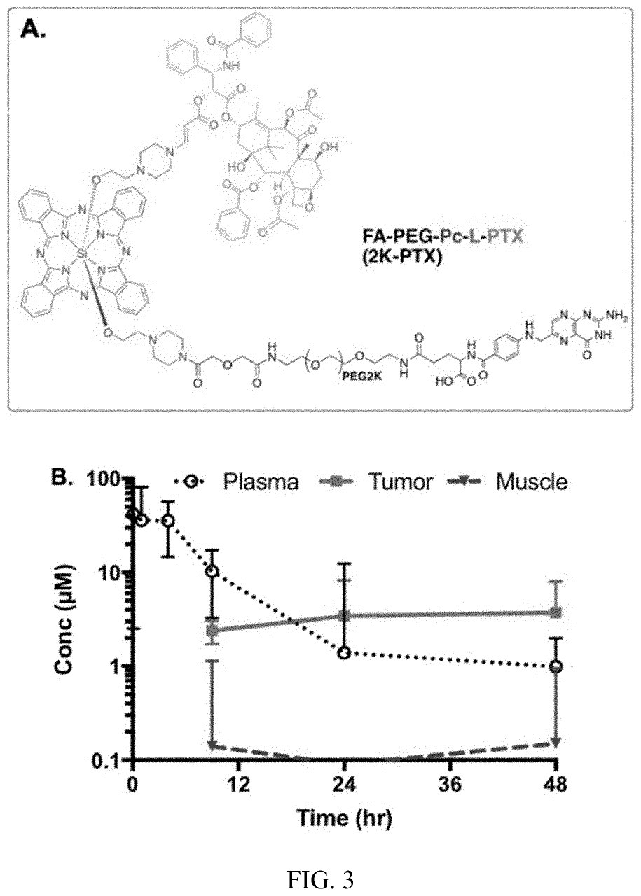

Prodrugs of PTX and CA4 were prepared, including: Pc-(L-CA4)₂, Pc-(L-PTX)₂, CA4-L-Pc-PEG-FA, and PTX-L-Pc-PEG-FA (a.k.a., 2K-PTX, see FIG. 3a) (MW_{PEG}=˜2K). Upon illumination with a 690-nm diode laser, these prodrugs can generate SO (causing immediate PDT damage) that consequently cleaves the linker, releasing anticancer drugs (causing sustained site-specific chemotherapeutic damage in the illuminated area (FIG. 1B). The site-specifically released anticancer drug overcomes the spatio-temporal limits of SO (PDT) (FIG. 2A). In both in vitro and in vivo studies, the prodrugs showed much better cytotoxic and antitumor effects than did their corresponding forms having a non-cleavable linker (NCL), i.e., Pc-(NCL-CA4)₂ and Pc-(NCL-PTX)₂. The PTX prodrug with the cleavable linker released at least about 90% of PTX upon illumination.

Folic Acid-Targeted PTX Prodrug [FA-PEG-Pc-L-PTX (2K-PTX)] Showed Preferential Uptake in FR-Expressing Cancer Cells (In Vitro) and Tumors (In Vivo)

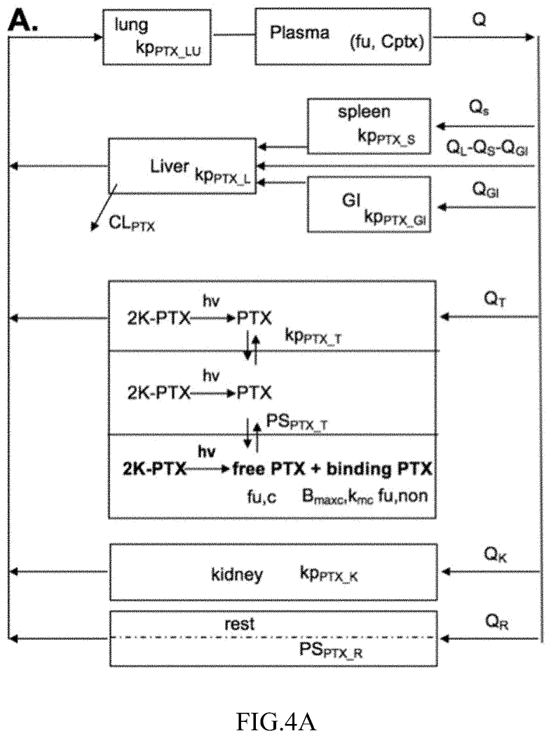

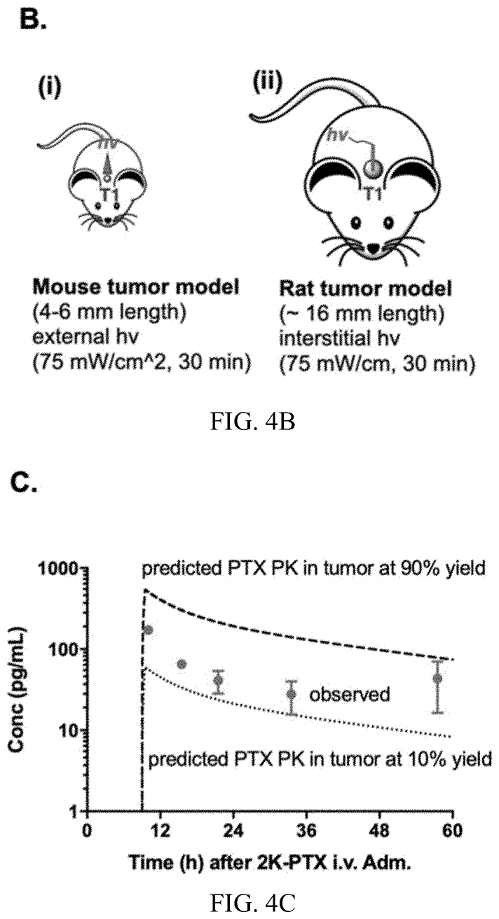

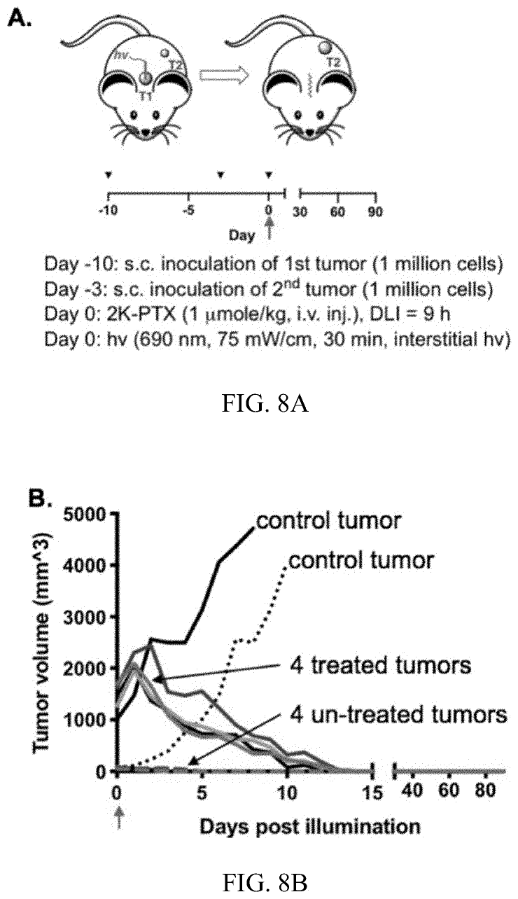

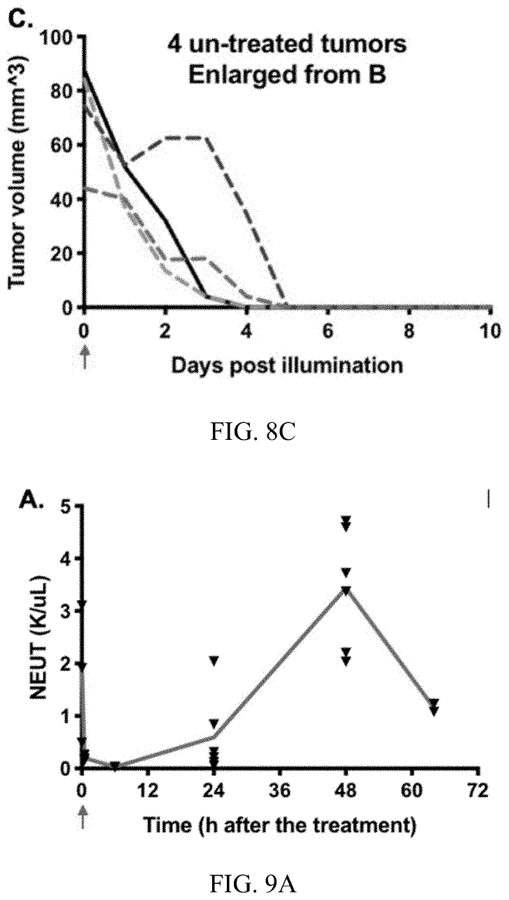

FR (folate receptor) is a demonstrated molecular target for anticancer drug delivery and whose expression correlates with aggressiveness and prognosis. TNBCs overexpress FR that can be used as a target of immunological agents, such as CAR-T cells and therapeutic antibodies. 2K-PTX showed FR-mediated uptake in various cancer cells (SKOV3, colon 26, and MDA-B-231) in vitro. Two syngeneic (“immunocompetent”) animal tumor models (Colon 26 tumor/balb/c mouse, a small tumor model (4-6 mm in length, volume ˜50 mm³), and MAT B III tumor/Fischer 344 rat), a large tumor model (˜16 mm in length, vol. ˜1500 mm³), were used. External beam illumination was used in the mouse model (FIG. 4Bi) and interstitial illumination (FIG. 4Bii) was used in the rat model. 2K-PTX showed preferential uptake to tumor vs. muscle [e.g., Conc.ₜᵤₘₒᵣ/Conc._{muscle}=17 (MAT B III, FIG. 3B) and 8.4 (colon 26) at 9 h post i.v. administration).

A PBPK Model of 2K-PTX was Established and Validated with the Experimental PK Data

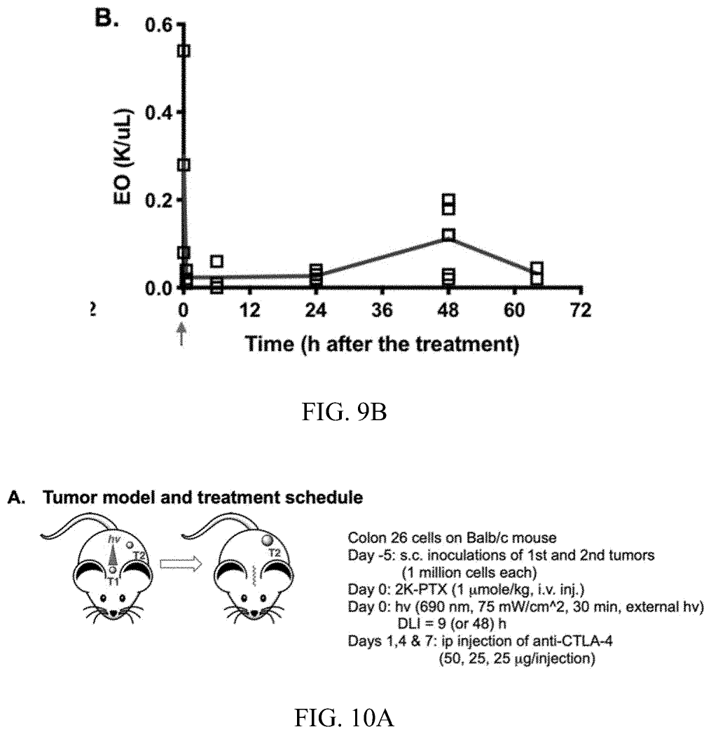

Physiologically-based pharmacokinetic (PBPK) modeling offers a number of valuable advantages. A well-established PBPK model enables prediction of pharmacokinetic (PK) profiles under different conditions, such as dose, administration route, and species, before time-consuming and resource-extensive experiments are performed. The predictive data can be used to rationally design such experiments. For these light-activated prodrugs, the data enabled us to simulate PK profiles of 2K-PTX in various organs at different doses and in different species (mouse, rat, dog, or human). Simulation of PK profiles of the released PTX in tumor and plasma after the illumination was also possible. FIGS. 9A=9B show pharmacodynamic profiles of neutrophils and eosinophils in blood, following the treatment with the prodrug (rat, 16 mm MAT B III s.c. tumor; 2K-PTX, 1 μmole/kg, DLI=9 h, 75 mW/cm, 30 min, interstitial illumination, 1 treatment on day 0; 3 (2 measurement/rat) rats/group) to treated and untreated tumors.

Based on our model, the released PTX PK profiles in tumors were predicted when cleavage yield is 90 or 10% (FIG. 4C). In the experimental data, the yield of PTX release by the illumination was ˜29% in the large rat tumor model with the interstitial illumination conditions. PBPK modeling also offers better insight into analyzing the data. Compared with the reported clearance rate of PTX from tumor, the clearance rate of the released PTX after the illumination was significantly slower after 24 h post-illumination (FIG. 4C). This result may have been in part a result of vascular damage/occlusion in the tumor by the PDT effect. Rapid vascular damage is a well-known PDT mechanism, along with direct cell killing and immune stimulation.

One Treatment with 2K-PTX Cured all Illuminated Tumors in Both the Small Tumor and Large Tumor Rodent Models

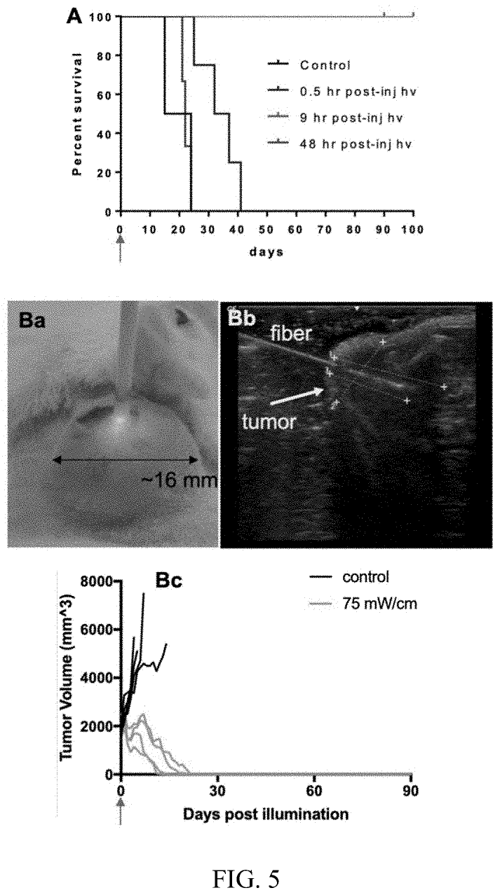

In PDT, drug-light interval (DLI, time between drug administration and illumination) is a key variable largely impacting antitumor mechanism and efficacy. Typically, short (e.g., ˜5-30 min) or long (48-72 h) DLI is used for PDT to achieve vascular-damaging PDT (V-PDT) or cytotoxic PDT (C-PDT). However, we chose a medium DLI (9 h), when both plasma and tumor have enough prodrug concentrations (FIG. 3b), to maximize antitumor effects via both V-PDT and C-PDT. We found that results with illumination at 9 h was better than illumination at 0.5 h or 48 h with the FA-PEG-Pc-L-PTX (2K-PTX) prodrug (FIG. 5a). With these treatment conditions, the tumors in all treated animals (4 animals per each group) were cured. No recurrence was observed at least for 90 days. The small tumor on the mouse was illuminated with a frontal diffuser (external beam); the large tumor on the rat was interstitially illuminated using a cylindrical diffuser (FIG. 4B).

In summary, a light-activatable prodrug platform using an SO-cleavable linker was used to develop prodrugs comprising CA4 and PTX, which showed a combined antitumor effect of rapid PDT effect and sustained and site-specific chemotherapeutic effect. PBPK models of PTX prodrug and released PTX after the illumination were established. Both small and large tumors were completely cured without recurrence for >90 days, without detectable systemic side effects. The large breast tumor was treated with ultrasound-image-guided fiber insertion (FIG. 5Bb), which demonstrated the feasibility of interstitial light therapy for treating deep-seated breast cancers under the thick human skin.

Without wishing to be held by theory, it is believed that (1) the local treatment with the prodrug produces a systemic effect to control cancers in the untreated area (via the abscopal effect-AE) a concept of “in-situ vaccination”, (2) the systemic effect depends on the treatment parameters, and also on the conditions of the cancers, (3) the systemic effect is further boosted by the combination with agents modulating the immune system, and (4) the whole cancer vaccine produced by the prodrug shows similar anticancer effects to the in-situ vaccine of the prodrug.

In one embodiment, the present disclosure is directed to use of multifunctional prodrugs for prevention of metastasis and recurrence, and for the treatment of metastatic breast cancers, with or without a combination with clinically available immunological agents.

In at least one embodiment, a light-activated prodrug is used to control both illuminated tumor and distant tumors via AE. In at least one other embodiment, a whole cell vaccine produced by the prodrug, is used alone or in combination with immune-modulating drugs or molecules. In clinical settings, the first will be applicable to metastatic and recurrent cancers, such as breast cancers, in the chest wall and other superficial areas where one of the tumors will be readily reachable for illumination either by external beam or an interstitial fiber. The prodrug vaccine therapy of the present disclosure is applicable to any metastatic condition, in particular it can be used in adjuvant settings, where tumor burden is relatively small, to replace adjuvant chemotherapy, thus minimizing toxicity from the chemotherapy treatment.

There has not been a systemic investigation about the “quantitative relationship” between basic pharmacological parameters (e.g., treatment conditions and disease burden/status) and AE. We hypothesized that AE has a typical pharmacological relationship (i.e., dose-response) with treatment- and tumor-related parameters. Experiments were designed to establish a quantitative relationship between these parameters with AE in the animal models to maximize the antitumor effects and to identify limitations (e.g., size and stage of tumors treatable by AE).

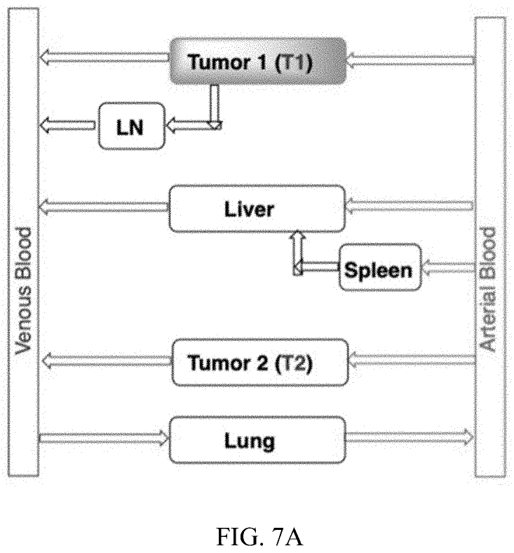

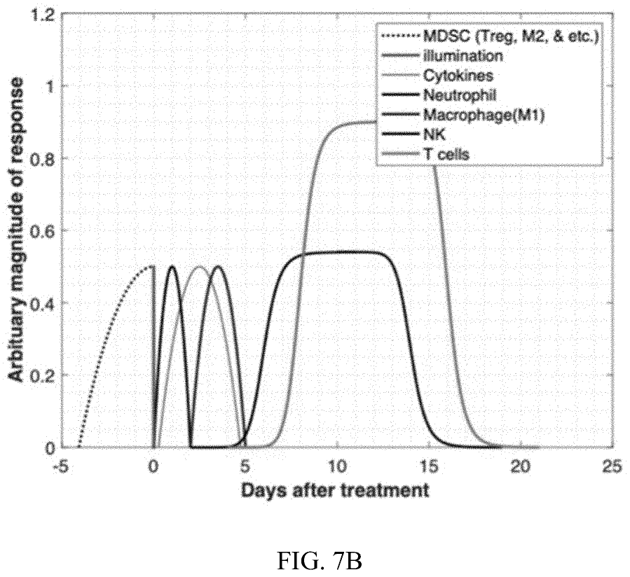

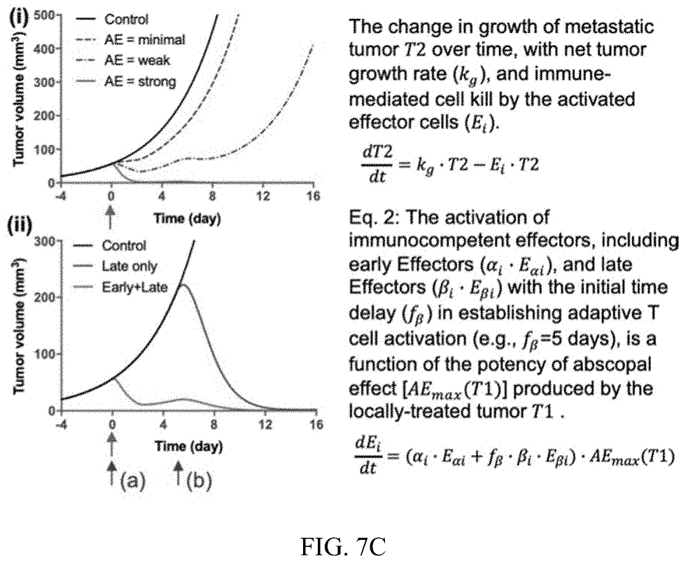

The AE by monotherapy (e.g., radiation or PDT) is more effective to prevent recurrence or metastasis when the tumor is small. However, the AE by monotherapy may not be strong enough to treat established large tumors. Since AE is mediated via the immune system, it was reasonable to hypothesize that AE can be potentiated by combination with immune-modulating agents. Recent dramatic advances in immune therapy offer a number of clinically available drugs for combination with the local therapies. Based on the pharmacodynamic resolution of data with typical dynamics of immune responses (see FIG. 7B vs. 8C), the AE from the prodrug is mediated not only by slow adaptive immune response, but also by fast innate immune responses.

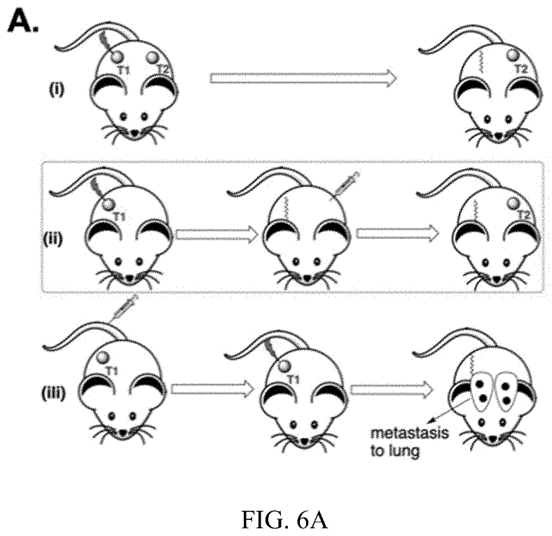

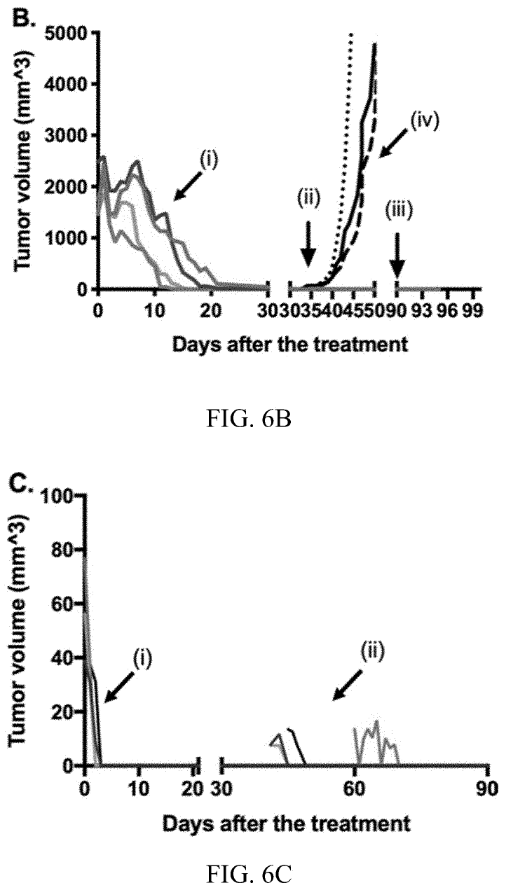

A tumor re-challenge model (FIG. 6Aii) was used to determine whether the adaptive immune system is activated to reject the re-challenged cancer cells. We first confirmed that tumor-bearing fisher rats do not reject re-challenged MAT B without tumor treatment. In results shown in FIGS. 6B-6C, all animals [rats (FIG. 6B) and mice (FIG. 6C)] whose first tumor was cured with our prodrug treatment rejected the re-challenged cancer cells 30˜90 days post-treatment (FIG. 6B).

Immunological agents affecting both innate and adaptive immune systems (e.g., cell-mediated) have been investigated for anticancer immunotherapy. Recent advances have focused more on the cell-mediated adaptive immune system (e.g., CD8+ T cells) to control metastatic tumors, which was also observed in PDT-mediated AE. However, the innate immune system (e.g., NK, macrophages, and neutrophils) has shown to play a key role in the anti-cancer immune response in PDT-“treated” tumors.

The results showed a rapid response to the second tumor (FIG. 8C), we consider that not only T cells, but also other mechanisms (e.g., innate immune cells), make contributions to the AE at different response rates (FIG. 7B). Our model simulations of antitumor effects by AE (FIG. 7Cii) support our hypothesis that the ‘early’ effectors that coincide with the timeframe of innate immune activation contribute to the AE, prior to the ‘late’ effectors that come into effect after the initial time delay in establishing adaptive immune T-cell activation.

In the present work, it was found treatment conditions ablated the small second tumor via the AE after treating the large first tumor (2K-PTX, 1 mole/kg, DLI: 9 hr, 690 nm, interstitial hv at 75 mW/cm for 30 min).

The results established a PBPK model of 2K-PTX and confirmed that the locally released PTX in the illuminated tumor is sufficient for cytotoxic cell kill (FIG. 4C, >50 nM for at least ˜60 h). PTX is also known to deplete Tregs and MDSCs.

The results demonstrated the rapid abscopal response in second tumors starting immediately (from day 1) following the first tumor treatment (FIG. 8C), which is much faster than typical rate of T cell-based immune response (1 day vs. >˜4-15 days, FIG. 6B).

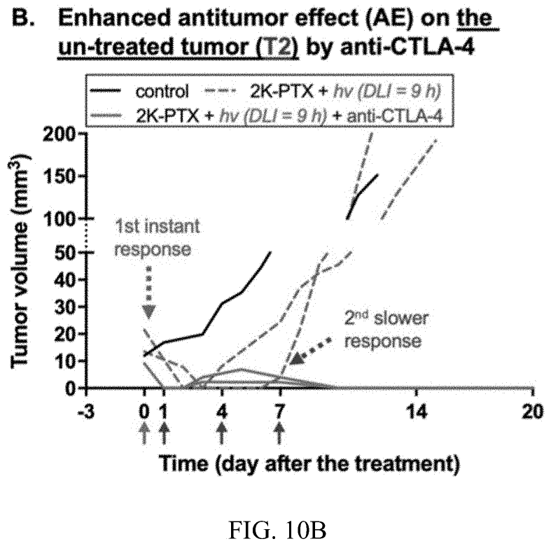

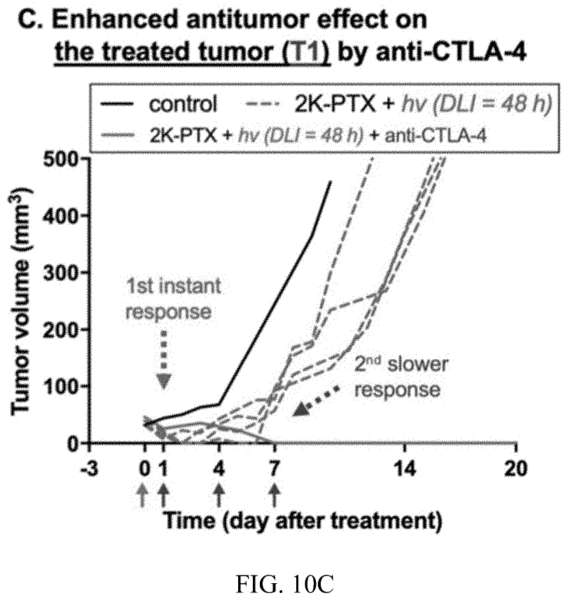

As noted, in one embodiment the prodrugs of the present disclosure may be used in adjunct (supplied before, concurrently, or after) with one or more immune therapy drugs to potentiate the AE of the prodrug for use in treating metastatic tumors or metastatic cells. For example, the prodrug could be administered with cabozantinib (e.g., with the cabozantinib supplied at 50 mg/kg, half of curative dose of monotherapy). The prodrug could be used in combination with other immune drugs that modulate adaptive or innate immune responses, such as, but not limited to, (1) checkpoint inhibitors (e.g., anti-CTLA-4, anti-PD-1, and anti-PD-L1) and (2) immune-modulating agents for stimulating innate immune cells (e.g., neutrophils), which play an important role in the fast response to 2K-PTX anti-tumor effect. For example, in one experiment, the addition of anti-CTLA-4 potentiated antitumor response by the late effector (FIGS. 10B-10C vs. FIG. 7C).

In another embodiment, the prodrugs of the used in the present disclosure (e.g., 2K-PTX) could be used to produce whole cell vaccine by treating the cells (derived for example from a subject's tumor, e.g., a breast cancer tumor) with the prodrug (e.g., wherein the cancer cells are killed by both PDT and PTX in vitro). The vaccine could be administered to the subject from whom the tumor cells are derived, with or without an immuno-modulating agent. Such a vaccine could be used as an adjuvant therapy following surgery, for example when the primary tumor could be completely removed, or incompletely removed, and/or as a treatment for metastatic cancers.

In one embodiment, the vaccine could be prepared using known methods for producing PDT-vaccines, such as by the methods of Gollnick (Roswell Park Cancer Institute) and Korbelik (British Columbia Cancer Agency), the pioneers in PDT-vaccines, using their established methods with minor modifications.

In at least one non-limiting embodiment, the present disclosure is directed to a method of treating or mitigating metastatic cancer in a subject, the subject having a primary tumor, wherein the method includes the steps of (a) providing a prodrug system, the prodrug system comprising (1) a prodrug comprising at least one functional moiety and at least one linker linked to the at least one functional moiety, the at least one linker selected from the group consisting of aminoacrylate, aminoacrylthioate, aminoacrylamide, and beta-aminoketone, wherein the at least one functional moiety is inactive when linked to the linker, and wherein the at least one linker is cleavable by singlet oxygen, and (2) a sensitizer which when exposed to an activator results in generation of singlet oxygen by the sensitizer, causing cleavage of the at least one linker thereby activating the at least one functional moiety, wherein the sensitizer optionally is linked to the functional moiety via the at least one linker; (b) administering the prodrug system to the subject, wherein the prodrug and sensitizer is allowed to accumulate in the primary tumor; and (c) exposing the primary tumor to the activator causing activation of the functional moiety and death of at least a portion of the primary tumor. In certain embodiments, the prodrug and the sensitizer are not linked, and the prodrug and the sensitizer are administered to the subject either simultaneously or sequentially. The prodrug may comprise a targeting moiety which preferentially binds to a receptor in the primary tumor. The targeting moiety may be selected from the group consisting of antibodies, ligands, tumor markers, aptamers, polyethylene glycol, albumin, tumor specific peptides, affibodies, vitamins, carbohydrates, hormones, low density lipoproteins (LDL), and combinations thereof. The prodrug system may a carrier moiety, which may be for example liposomes, polymers, nanospheres, nanocapsules, micelles, solid lipid nanoparticles, or combinations thereof. The prodrug may be in the form of a dendrimer. The sensitizer may be a photosensitizer selected from the group consisting of porphyrin, phthalocyanines, boron-dipyrromethene (BODIPY) or aza-BODIPY-type photosensitizers, chlorins, bacteriochlorins, non-porphyrin-based photosensitizers, and combinations thereof. The prodrug may comprise a spacer between the linker and the sensitizer and/or between the linker and the functional moiety. The spacer may be selected from the group consisting of piperidin-4-ylmethanol, pyrrolidine-2-carboxylic acid, pyrrolidine-3-carboxylic acid, piperidin-4-ylmethyl-2-bromoacetate, 1-(3-bromoporpyl)piperazine, and combinations thereof. The at least one functional moiety may be a therapeutic moiety and/or a detectable moiety. The prodrug may comprise two or more functional moieties, wherein the two or more functional moieties are the same or are different molecules. The activator may be selected from the group consisting of irradiation with visible/near IR light, irradiation with ionizing radiation, exposure to electromagnetic waves/materials, exposure to luminescence, exposure to fluorescence, and combinations thereof. The activator may comprise irradiation with light in a range of from about 380 nm to about 1200 nm. The method may further comprise providing an adjunct therapy before, concurrently with, or after, administering the prodrug system.

In another nonlimiting embodiment, the disclosure is directed to a method of making a whole-cell cancer vaccine, comprising the steps of (a) providing live cancer cells obtained from a tumor of a subject; (b) providing a prodrug system, the prodrug system comprising (1) a prodrug comprising at least one functional moiety and at least one linker linked to the at least one functional moiety, the at least one linker selected from the group consisting of aminoacrylate, aminoacrylthioate, aminoacrylamide, and beta-aminoketone, wherein the at least one functional moiety is inactive when linked to the linker, and wherein the at least one linker is cleavable by singlet oxygen, and (2) a sensitizer which when exposed to an activator results in generation of singlet oxygen by the sensitizer, causing cleavage of the at least one linker thereby activating the at least one functional moiety, wherein the sensitizer optionally is linked to the functional moiety via the at least one linker; (c) treating the live cancer cells with the prodrug system; and (d) exposing the treated live cancer cells to the activator thereby causing activation of the functional moiety and death of the live cancer cells to form the whole cell cancer vaccine. The prodrug may further comprise a targeting moiety which preferentially binds to a receptor in the primary tumor. The targeting moiety may be selected from the group consisting of antibodies, ligands, tumor markers, aptamers, polyethylene glycol, albumin, tumor specific peptides, affibodies, vitamins, carbohydrates, hormones, low density lipoproteins (LDL), and combinations thereof. The prodrug may be in the form of a dendrimer. The sensitizer may be a photosensitizer selected from the group consisting of porphyrin, phthalocyanines, boron-dipyrromethene (BODIPY) or aza-BODIPY-type photosensitizers, chlorins, bacteriochlorins, non-porphyrin-based photosensitizers, and combinations thereof. The prodrug may further comprise a spacer between the linker and the sensitizer and/or between the linker and the functional moiety. The spacer may be selected from the group consisting of piperidin-4-ylmethanol, pyrrolidine-2-carboxylic acid, pyrrolidine-3-carboxylic acid, piperidin-4-ylmethyl-2-bromoacetate, 1-(3-bromoporpyl)piperazine, and combinations thereof. The prodrug may be further defined as comprising two or more functional moieties, wherein the two or more functional moieties are the same or are different molecules. The activator may be selected from the group consisting of irradiation with visible/near IR light, irradiation with ionizing radiation, exposure to electromagnetic waves/materials, exposure to luminescence, exposure to fluorescence, and combinations thereof. The activator may comprise irradiation with light in a range of from about 380 nm to about 1200 nm. Other aspects of the prodrug system are as shown above.

In another non-limiting embodiment, the present disclosure is directed to a method of treating or mitigating metastatic cancer in a subject, the method comprising administering to the subject the whole cell cancer vaccine made from live cancer cells from the subject by using the method described above. In the method, the live cancer cells may have been obtained from a primary tumor completely or substantially removed from the subject prior to the subject being treated with the whole cell vaccine. The method may further comprise providing an adjunct therapy to the subject before, concurrently with, or after, administering the whole cell vaccine to the subject.

Further explanation of the embodiments of the present disclosure, and description of other embodiments of the present disclosure, are provided in Appendix 1 of U.S. Ser. No. 62/815,487, which is explicitly incorporated herein by reference in its entirety.

It will be understood from the foregoing description that various modifications and changes may be made in the various embodiments of the present disclosure without departing from their true spirit. The description provided herein is intended for purposes of illustration only and is not intended to be construed in a limiting sense. Thus, while embodiments of the present disclosure have been described herein so that aspects thereof may be more fully understood and appreciated, it is not intended that the present disclosure be limited to these particular embodiments. On the contrary, it is intended that all alternatives, modifications and equivalents are included within the scope of the inventive concepts as defined herein. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulations and compositions described herein, the methods described herein or in the steps or the sequence of steps of the methods described herein without departing from the spirit and scope of the present disclosure.

What is claimed is:

1. A method of treating or mitigating metastatic cancer in a subject, the subject having a primary tumor, the method comprising:
providing a prodrug system, the prodrug system comprising (1) a prodrug comprising at least one functional moiety and at least one linker linked to the at least one functional moiety, the at least one linker selected from the group consisting of aminoacrylate, aminoacrylthioate, aminoacrylamide, and beta-aminoketone, wherein the at least one functional moiety is inactive when linked to the linker, and wherein the at least one linker is cleavable by singlet oxygen, and (2) a sensitizer which when exposed to an activator results in generation of singlet oxygen by the sensitizer, causing cleavage of the at least one linker thereby activating the at least one functional moiety, wherein the sensitizer optionally is linked to the functional moiety via the at least one linker;
administering the prodrug system to the subject, wherein the prodrug and sensitizer is allowed to accumulate in the primary tumor; and
exposing the primary tumor to the activator causing activation of the functional moiety and death of at least a portion of the primary tumor.

2. The method of claim 1, wherein the prodrug and the sensitizer are not linked, and the prodrug and the sensitizer are administered to the subject either simultaneously or sequentially.

3. The method of claim 1, wherein the prodrug further comprises a targeting moiety which preferentially binds to a receptor in the primary tumor.

4. The method of claim 3, wherein the targeting moiety is selected from the group consisting of antibodies, ligands, tumor markers, aptamers, polyethylene glycol, albumin, tumor specific peptides, affibodies, vitamins, carbohydrates, hormones, low density lipoproteins (LDL), and combinations thereof.

5. The method of claim 1, wherein the prodrug system further comprises a carrier moiety.

6. The method of claim 5, wherein the carrier moiety is selected from the group consisting of liposomes, polymers, nanospheres, nanocapsules, micelles, solid lipid nanoparticles, and combinations thereof.

7. The method of claim 1, wherein the prodrug is further defined as being in the form of a dendrimer.

8. The method of claim 1, wherein the sensitizer is a photosensitizer selected from the group consisting of porphyrin, phthalocyanines, boron-dipyrromethene (BODIPY) or aza-BODIPY-type photosensitizers, chlorins, bacteriochlorins, non-porphyrin-based photosensitizers, and combinations thereof.

9. The method of claim 1, wherein the prodrug further comprises a spacer between the linker and the sensitizer and/or between the linker and the functional moiety.

10. The method of claim 9, wherein the spacer is selected from the group consisting of piperidin-4-ylmethanol, pyrrolidine-2-carboxylic acid, pyrrolidine-3-carboxylic acid, piperidin-4-ylmethyl-2-bromoacetate, 1-(3-bromoporpyl)piperazine, and combinations thereof.

11. The method of claim 1, wherein the at least one functional moiety is a therapeutic moiety and/or a detectable moiety.

12. The method of claim 1, wherein the prodrug is further defined as comprising two or more functional moieties, wherein the two or more functional moieties are the same or are different molecules.

13. The method of claim 1, wherein the activator is selected from the group consisting of irradiation with visible/near IR light, irradiation with ionizing radiation, exposure to electromagnetic waves/materials, exposure to luminescence, exposure to fluorescence, and combinations thereof.

14. The method of claim 1, wherein the activator comprises irradiation with light in a range of from about 380 nm to about 1200 nm.

15. The method of claim 1, further comprising providing an adjunct therapy before, concurrently with, or after, administering the prodrug system.

16. A method of making a whole-cell cancer vaccine, comprising:
providing live cancer cells obtained from a tumor of a subject;
providing a prodrug system, the prodrug system comprising (1) a prodrug comprising at least one functional moiety and at least one linker linked to the at least one functional moiety, the at least one linker selected from the group consisting of aminoacrylate, aminoacrylthioate, aminoacrylamide, and beta-aminoketone, wherein the at least one functional moiety is inactive when linked to the linker, and wherein the at least one linker is cleavable by singlet oxygen, and (2) a sensitizer which when exposed to an activator results in generation of singlet oxygen by the sensitizer, causing cleavage of the at least one linker thereby activating the at least one functional moiety, wherein the sensitizer optionally is linked to the functional moiety via the at least one linker;
treating the live cancer cells with the prodrug system; and
exposing the treated live cancer cells to the activator thereby causing activation of the functional moiety and death of the live cancer cells to form the whole cell cancer vaccine.

17. The method of claim 16, wherein the prodrug further comprises a targeting moiety which preferentially binds to a receptor in the primary tumor.

18. The method of claim 17, wherein the targeting moiety is selected from the group consisting of antibodies, ligands, tumor markers, aptamers, polyethylene glycol, albumin, tumor specific peptides, affibodies, vitamins, carbohydrates, hormones, low density lipoproteins (LDL), and combinations thereof.

19. The method of claim 16, wherein the prodrug is further defined as being in the form of a dendrimer.

20. The method of claim 16, wherein the sensitizer is a photosensitizer selected from the group consisting of porphyrin, phthalocyanines, boron-dipyrromethene (BODIPY) or aza-BODIPY-type photosensitizers, chlorins, bacteriochlorins, non-porphyrin-based photosensitizers, and combinations thereof.

21. The method of claim 16, wherein the prodrug further comprises a spacer between the linker and the sensitizer and/or between the linker and the functional moiety, and wherein the spacer is selected from the group consisting of piperidin-4-ylmethanol, pyrrolidine-2-carboxylic acid, pyrrolidine-3-carboxylic acid, piperidin-4-ylmethyl-2-bromoacetate, 1-(3-bromoporpyl)piperazine, and combinations thereof.
